(19) United States
(12) Patent Application Publication
(10) Pub. No.: US 2019/0046748 A1
(43) Pub. Date: Feb. 14, 2019

(54) ILMA ASSEMBLY HAVING DIVIDED SHEATH

(71) Applicants: Aaron McClellan, Lebanon, NH (US); Ryan C. Stockett, Lebanon, NH (US)

(72) Inventors: Aaron McClellan, Lebanon, NH (US); Ryan C. Stockett, Lebanon, NH (US)

(21) Appl. No.: 15/675,655

(22) Filed: Aug. 11, 2017

(51) Int. Cl. A61M 16/04

(52) U.S. Cl. CPC A61M 16/0402; A61M 25/01; A61M 16/0465; A61M 16/0434

(57) ABSTRACT

A laryngeal mask airway is disclosed for use with an endotracheal tube. The laryngeal mask air may include a tubular sheath having an opening extending from a first end to a second end. The laryngeal mask airway may also include a mask located at the second end. The laryngeal mask airway may be split axially into two sides that are removably connected to each other along their length.

### TECHNICAL FIELD

The present disclosure relates generally to an intubating laryngeal mask airway (ILMA) assembly and, more particularly, to an ILMA assembly having a divided sheath.

### BACKGROUND

A laryngeal mask airway (LMA) is a super-glottis device, which includes a latex mask connected to an end of a hollow plastic tube. The LMA is inserted, mask-first, into the lower pharynx of a patient and over the glottis. In some configurations, the mask includes a cuff that can be inflated so as to force the mask into the hypopharynx. This causes the mask to seal around the laryngeal inlet. The LMA can be used to open the patient's airway, and functions as a temporary ventilation means.

An endotracheal tube (ETT) is a plastic tube that is inserted through the mouth and into the trachea of a patient, in order to maintain ventilation/oxygenation during extended surgeries or other airway emergencies. During insertion of the ETT, care must be taken to avoid lacerating throat tissues, the vocal cords, and/or the upper airway. Conventionally, a scope (e.g., a laryngoscope, a fiber-optic bronchoscope, and/or a video laryngoscope) is used to thread the tube between the vocal cords and into the trachea.

In some situations, a scope may not be available and/or its use may be too slow. In these and other instances, the LMA can be used as a guide for blindly inserting the ETT. For example, the LMA can be inserted into the airway of an unconscious patient until the mask is seated in the hypopharynx. At this point in time, the patient is apneic. The ETT is then passed through the LMA (e.g., blindly or with the use of a scope) until a tip end of the tube passes through the vocal cords and into the trachea. A balloon located at a tip end of the ETT is then inflated, and placement of the ET in the trachea is confirmed. Standard practice is then to remove the LMA after intubation (e.g., due to a risk of mucosal damage to the posterior hypopharynx. The ETT is held in a stationary position, while the LMA is withdrawn from the airway. The ETT is then secured and ventilation is resumed.

Although successful in most instances, the use of an ETT together with an LMA may pose significant risk to the unconscious patient. A primary concern is the amount of time during which the patient is apneic during placement of the LMA/ETT and during removal of the LMA. Another concern is accidental extubation of the ETT upon removal of the LMA. In addition, tissue damage can occur from inadvertently pushing the ETT too far into the trachea (e.g., using a stabilizing rod) during removal of the LMA.

The ILMA assembly of the present disclosure solves one or more of the problems set forth above and/or other problems in the art.

### SUMMARY

In one aspect, the present disclosure is directed to a laryngeal mask airway for use with an endotracheal tube. The laryngeal mask air may include a tubular sheath having an opening extending from a first end to a second end. The laryngeal mask airway may also include a mask located at the second end. The laryngeal mask airway may be split axially into two sides that are removably connected to each other along their length.

In another aspect, the present disclosure is directed to an intubating laryngeal mask airway assembly. The intubating laryngeal mask airway assembly may include a laryngeal mask airway having two sides that are removably connected to each other along their length. The intubating laryngeal mask airway may also include an endotracheal tube disposed inside of the laryngeal mask airway prior to separation of the two sides.

In yet another aspect, the present disclosure is directed to a method of intubating a patient. The method may include placing an intubating laryngeal mask airway assembly, including a laryngeal mask airway and an endotracheal tube, into a lower pharynx of the patient. The method may also include separating the laryngeal mask airway into multiple components, and independently removing each of the components of the laryngeal mask airway from the patient while the endotracheal tube remains in place.

### BRIEF DESCRIPTION OF THE DRAWING

FIGS. 1 and 2 are front- and side-view illustrations, respectively, of an exemplary disclosed intubating laryngeal mask airway (ILMA) assembly;

FIG. 3 is an exploded view illustration of the ILMA assembly of FIGS. 1 and 2;

FIGS. 4-6 are cross-sectional view illustrations of different portions of the ILMA assembly of FIGS. 1-3;

FIG. 7 is a flow diagram of an exemplary method of placing the ILMA assembly of FIGS. 1-3 in the airway of a patient during intubation; and

FIGS. 8 and 9 are additional cross-sectional view illustrations of the portion of the ILMA assembly shown in FIG. 6.

### DETAILED DESCRIPTION

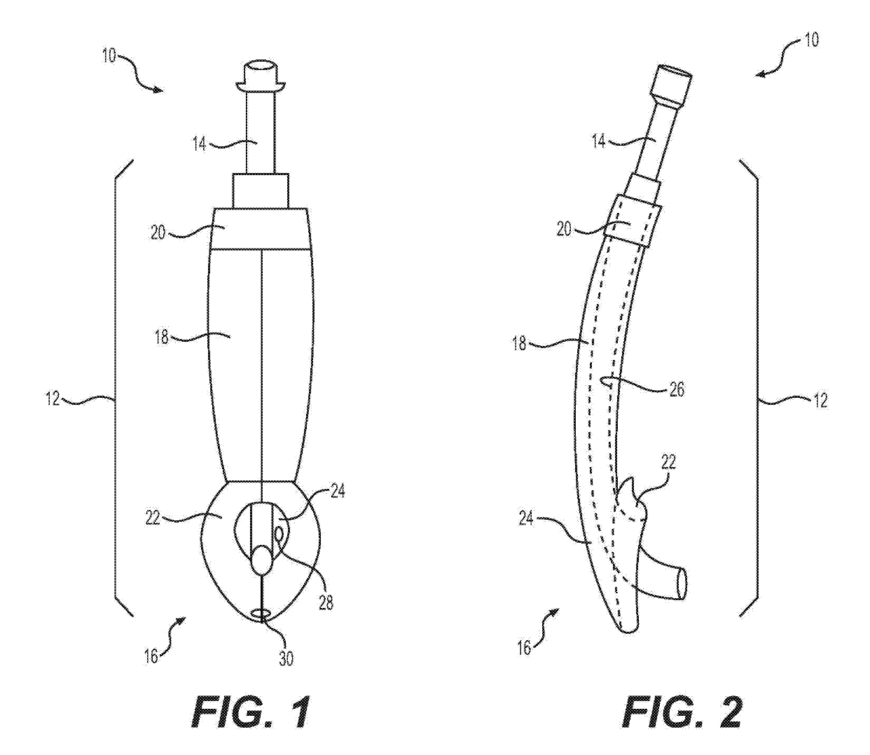

FIGS. 1 and 2 illustrate an exemplary ILMA assembly 10 having an LMA 12 and an ETT 14. ETT 14 may be slideably disposed in LMA 12 and packaged together as an assembled kit. It is contemplated, however, that LMA 12 and ETT 14 could be packaged separately and used together or separately, as desired.

LMA 12 may generally comprise a mask 16, a sheath 18 extending away from mask 16, and an endcap or collar 20 connectable to sheath 18 at a second end opposite mask 16. Mask 16 may include an annular cuff 22 that may be solid or hollow and inflatable, and a hood 24 that encloses one side of cuff 22 and is fixedly connected to sheath 18. Cuff 22 and/or hood 24 may be generally flexible, available in multiple sizes and shapes, and able to conform to the contours of a patient's throat.

Sheath 18 may be generally tubular, having one or more openings formed therein that extend from the mask-end through the second end. For example, sheath 18 may have a central opening 26 that is configured to function as an airway and/or as a conduit for receiving ETT 14. In addition, sheath 18 may include one or more peripheral openings (e.g., first and second side openings 28, 30 located opposite each other—see FIG. 6) that function as conduits for scopes, catheters, anesthesia injections, etc. Peripheral openings 28 and/or 30 may have smaller diameters (e.g., the same or different from each other) than central opening 26, and may extend from the second end of sheath 18 only as far as central opening 26 or further into hood 24 and/or cuff 22. Many different arrangements may be possible. Sheath 18 may have a circular cross-section, an oval cross-section, an ellipsoid cross-section, or another shape of cross-section, depending on the number and/or arrangement of internal openings. Sheath 18 may be somewhat flexible (e.g., flexible, but more rigid than cuff 22 and hood 24), or rigid. A handle, balloon pump, and/or other accessory (not shown) may be connected to or otherwise extend through sheath 18, if desired.

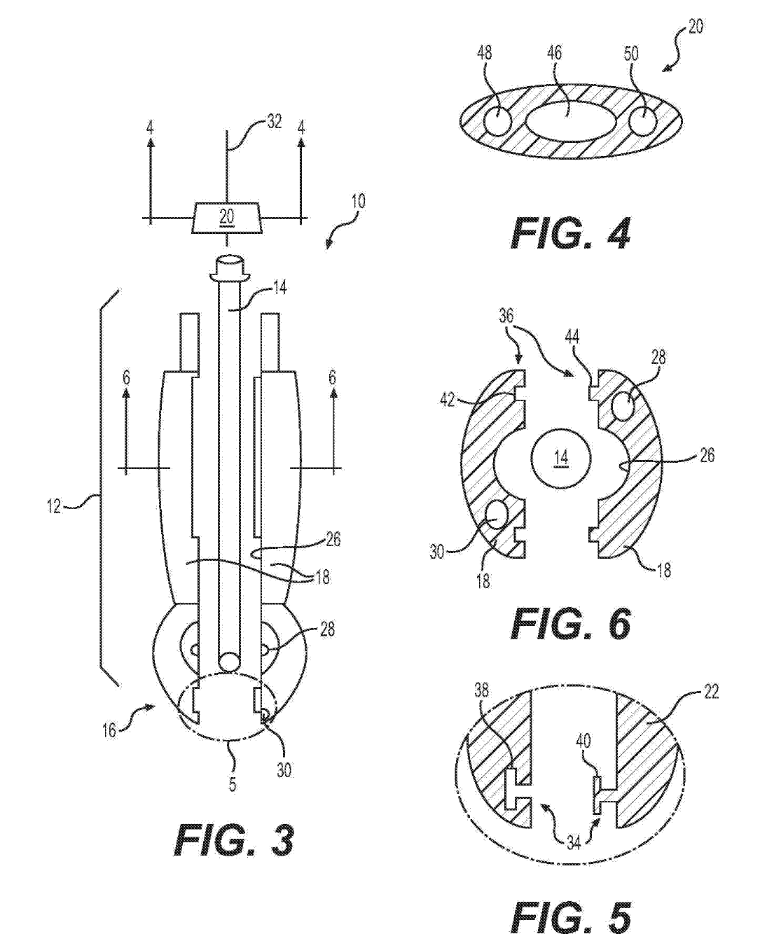

In the embodiment shown in FIG. 3, LMA 12 may be split into two sides that are removably connected to each other (i.e., connected in such a way that removal is possible without causing destruction). The parts are substantially identical or mirror images of each other (e.g., other than associated connection features described below), and separable along a lengthwise-oriented plane 32 that passes symmetrically through an axis of central opening 26 (and through axes of peripheral openings 26 and 28, in some embodiments—not shown). In other embodiments, however, the separation of LMA 12 may be asymmetrical, such that one of openings 26 and 28 is located within each part (see FIG. 5). Other configurations may also be possible. The two parts of LMA 12 may remain connected to each other during insertion of ILMA assembly 10 into the patient's throat, and then be disconnected for independent removal from the patient's throat after balloon inflation of ETT 14.

The two parts of LMA portion 12 may be connected to each other in many different ways. In the disclosed embodiment of FIGS. 3-6, each part of LMA 12 has at least one interlocking feature (e.g., a female feature) configured to engage a complimentary interlocking feature (e.g., a male feature) in the other part. The interlocking features may allow for relative sliding motion in the axial direction (i.e., along plane 32) and/or in an orthogonal direction (e.g., in a direction normal to plane 32).

In one example, two sets of interlocking features are provided within each part of LMA 12, including a lower set 34 (shown in detail in FIG. 5) associated with mask 16 and an upper set 36 (shown in detail in FIG. 6) associated with sheath 18. In this example, lower set 34 consists of a T-shaped channel or groove 38 formed within one part of LMA 12 at one side of cuff 22, and a corresponding T-shaped protrusion 40 formed within the other part of LMA 12. In this configuration, mask 16 may be separated into the two parts only by relative sliding in the axial direction, allowing protrusion 40 to be pushed into or pulled out of channel 38. In this configuration, lower set 34 may not be separated by movement in the orthogonal direction. This limitation may help to avoid unintended separation during installation or removal of LMA 12. It should be noted that other shapes (e.g., C- or Y-shapes) may be used, if desired.

Upper set 36 may consist of a simpler U-, V-, or bulbous-shaped channel or groove 42 formed within one part of sheath 18 at one edge thereof, and a corresponding inverted U-, V-, or bulbous-shaped protrusion 44 formed within the other part of sheath 18 at the same edge. Accordingly, each part of sheath 18 may have one (or more) channel 42 and one (or more) protrusion 44 at opposing edges, relative to central opening 26. In this configuration, sheath 18 may be separated into the two parts by relative sliding in either (or both) the axial and orthogonal directions. That is, upper set 36 may be separated by a pulling movement in the axial and/or in the orthogonal directions. This may allow upper set 36 to be assembled and disassembled at the same time as, before, and/or after lower set 34 during installation and/or removal of LMA 12.

Collar 20 may function to inhibit separation of LMA 12. In particular, collar 20 may be hollow, have an internal shape that generally conforms to the external shape of sheath 18, and be configured to pass over the second end of sheath 18 after the two parts of sheath 18 have been joined together. In one embodiment, a press-fit may keep collar 20 in place on sheath 18. In other embodiments, however, one or more engagement features (e.g., ridges, grooves, etc.) may be utilized for this purpose. As shown in FIG. 4, end cap 20 may include a central opening 46 configured to allow passage of ETT 14, and peripheral openings 48, 50 generally aligned with peripheral openings 28, 30 in sheath 18. Although collar 20 is shown and described as a separate component, it is contemplated that collar 20 could be integrally formed with one of the parts of sheath 18, if desired.

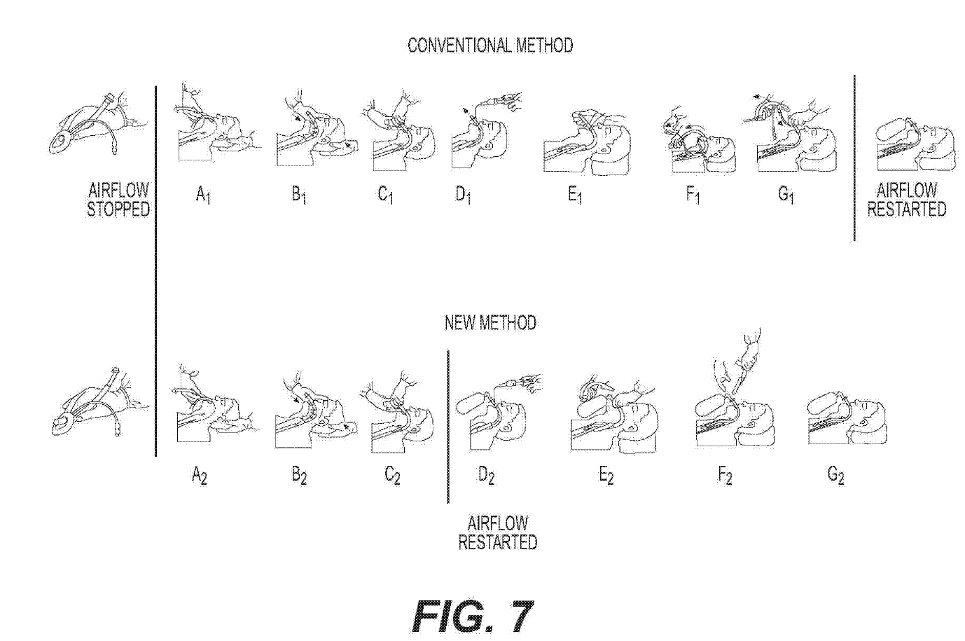

FIG. 7 illustrates exemplary intubation methods. FIG. 7 will be discussed in more detail in the following section to further illustrate the disclosed concepts.

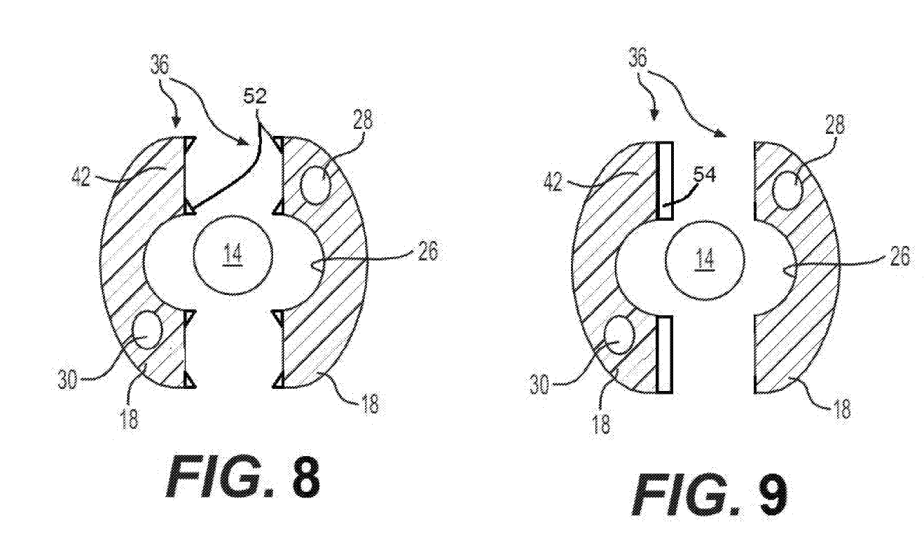

FIGS. 8 and 9 illustrate alternative ways in which the two parts of LMA portion 12 may be joined together. In the embodiment of FIG. 8, the two parts of LMA portion 12 are initial formed together as a single integral unit, each part having one or more narrowing protrusions (e.g., an inverted V-shaped ridge) 52 that are joined at tip-ends to similar narrowing protrusions of the adjacent part. Due to the narrowness of protrusions 52 at the joint interface, separation of the two parts may be achieved via pulling apart of the tip ends. It is contemplated that a single protrusion 52 may be used and located at a general center of each face of the joint or that multiple protrusions 52 (e.g., two) may be used and located at outer edges of each face. Each protrusion 52 may extend lengthwise a discrete distance or entirely around each part of LMA portion 12. It is also contemplated that protrusion(s) 52 may be used only at the upper end of LMA portion 12 (e.g., in conjunction with lower set 34), only at the lower end of LMA portion 12 (e.g., in conjunction with upper set 36), or both at the upper and lower ends. It is further contemplated that, rather than the two parts of the embodiment shown in FIG. 8 being integrally formed, the two parts could alternatively be formed separately and thereafter joined (e.g., via a chemical and/or mechanical bonding processes).

FIG. 9 illustrates an embodiment where each part of LMA portion 12 are formed separately and thereafter joined together. In this example, the two parts are joined together chemically, for example via double sided tape. As in the embodiment of FIG. 8, the two parts of LMA portion 12 may be separated via pulling of the parts in opposing directions.

### INDUSTRIAL APPLICABILITY

The disclosed ILMA assembly may be used to ventilate a patient in preparation for surgery and/or during an emergency. The disclosed ILMA assembly may allow for ventilation in less time and with few steps than conventional intubation. Use of the disclosed ILMA assembly and comparison with conventional intubation will now be described in detail, with reference to FIG. 7.

As shown in FIG. 7, conventional intubation may be performed in about seven different steps (A₁ through G₁), during which the patient's airway is blocked and the patient is unable to breath. In particular, the first step is labeled in FIG. 7 as A₁, and may include grasping a conventional LMA and inserting the LMA cuff-first into a patient's mouth. The second step is labeled as B₁, and may include pushing the LMA back against the hard palate. The LMA is then slid backwards following the curvature of the LMA, and swung into place—step C₁. The cuff is then inflated to inhibit removal of the LMA—step D₁.

After confirming proper placement of the LMA, the next step is labeled as E₁ and includes lubricating the ETT and passing it through the central tube of the LMA and into the trachea. A balloon of the ETT is then inflated to inhibit removal. Intubation is then complete, and the LMA can be removed.

Conventional removal of the LMA is performed at step F₁, and may include deflation of the cuff and insertion of a stabilizing rod into the ETT. Thereafter, the LMA may be pulled rearward over the ETT and stabilizing rod—step G₁. A ventilator may then be connected to the ETT, and ventilation may finally begin.

As also shown in FIG. 7, a new method of intubation utilizing the disclosed ILMA assembly may be performed with fewer steps (A₂ through G₂) and in less time during which the patient is unable to breath. The first three steps labeled in FIG. 7 as A₂, B₂, and C₂ may be substantially identical to steps A₁, B₁, and C₁ of the conventional method described above. However, ETT 14 may already be installed within LMA 12 prior to completion of these steps. In contrast to the conventional method described above, in the new method disclosed in this application, ventilation may begin immediately after completion of step C₂. Step D₂ may then be completed in the same way as step D₁, and at the same time as ventilating the patient.

After completion of step D₂, LMA 12 may be removed. It should be noted that LMA 12 may be removed simultaneous with patient ventilation. In fact, after ventilation begins at step D₂, ventilation may continue substantially uninterrupted. LMA 12 may be removed by first separating collar 20 from sheath 18. This separation may allow one side of sheath 18 at the upper end to be pulled apart from the opposing side. For example, the separation may allow the U- or V-shaped protrusion 44 of upper set 36 to be pulled in an orthogonal direction (relative to plane 32) out of the U- or V-shaped groove 42. Thereafter, the two sides of sheath 18 at the lower end may be slide in opposing axially directions to complete the severance of the two sides. For example, T-shaped protrusion 40 may be slid axially out of T-shaped groove 38. Once the two sides of sheath 18 are completely separated, each side may be independently removed from the patient's throat—steps E₂ and F₂.

As can be seen by a comparison of the conventional method and the new method, the ability to separate LMA 12 into multiple components allows for a much shorter period of time during which the patient is left apneic. In addition, the likelihood of accidental extubation may be reduced, as the separate side removals of LMA 12 may generate fewer and lower forces on ETT 14 during removal. Finally, a stabilizing rod may not be required during removal of LMA 12, thereby reducing the likelihood of tissue damage from pushing ETT 14 too far into the trachea.

It will be apparent to those skilled in the art that various modifications and variations can be made to the disclosed ILMA assembly. Other embodiments of the bed and hub will be apparent to those skilled in the art from consideration of the specification and practice of the ILMA assembly disclosed herein. It is intended that the specification and examples be considered as exemplary only, with a true scope of the disclosure being indicated by the following claims and their equivalents.

What is claimed is:

1. A laryngeal mask airway for use with an endotracheal tube, comprising:
a tubular sheath having a first end an extending from a first end to a second end; and
a mask located at the second end,
wherein the laryngeal mask airway is split axially into two sides that are removably connected to each other along their length.

2. The laryngeal mask airway of claim 1, further including at least one interlocking feature formed within each of the two sides that are configured to engage each other.

3. The laryngeal mask airway of claim 2, wherein the at least one interlocking feature includes:
a first set of interlocking features located at the first end of the tubular sheath; and
a second set of interlocking features located at the second end of the tubular sheath.

4. The laryngeal mask airway of claim 3, wherein the first set of interlocking features are different than the second set of interlocking features.

5. The laryngeal mask airway of claim 4, wherein:
the interlocking features of the first set are separable in either an axial direction or an orthogonal direction; and
the interlocking features of the second set are separable in only an axial direction.

6. The laryngeal mask airway of claim 5, wherein:
the interlocking features of the first set are integral with the tubular sheath; and
the interlocking features of the second set are integral with the mask.

7. The laryngeal mask airway, of claim 6, wherein:
the mask includes an annular cuff, and a hood that encloses one side of the annular cuff; and
the interlocking features of the second set are integral with the annular cuff.

8. The laryngeal mask airway of claim 1, wherein:
the two sides are separable along a lengthwise-oriented plane; and
the lengthwise-oriented plane passes symmetrically through the tubular sheath.

9. The laryngeal mask airway of claim 1, wherein:
the tubular sheath further includes at least one peripheral opening that extends from the first end to the second end; and
the lengthwise-oriented plane passes asymmetrically through the tubular sheath such that only one of the two sides includes the at least one peripheral opening.

10. The laryngeal mask airway of claim 1, further including a collar configured to engage the first end of the tubular sheath and inhibit separation of the two sides.

11. The laryngeal mask airway of claim 1, wherein the two sides are connected to each other via at least one of double-sided tape and tip ends of narrowing protrusions that extend from the two sides towards each other.

12. An intubating laryngeal mask airway assembly, comprising:
a laryngeal mask airway having two sides that are removably connected to each other along their length; and
an endotracheal tube disposed inside of the laryngeal mask airway prior to separation of the two sides.

13. The intubating laryngeal mask airway assembly of claim 12, wherein the laryngeal mask airway includes:
a tubular sheath having a first end an extending from a first end to a second end;
a mask located at the second end; and
at least one interlocking feature formed within each of the two sides that are configured to engage each other.

14. The intubating laryngeal mask airway assembly of claim 13, wherein the at least one interlocking feature includes:
a first set of interlocking features located at the first end of the tubular sheath and that are separable in either an axial direction or an orthogonal direction; and
a second set of interlocking features located at the second end of the tubular sheath and that are separable in only an axial direction.

15. The intubating laryngeal mask airway assembly of claim 13, further including a collar configured to engage the first end of the tubular sheath and inhibit separation of the two sides.

16. A method of intubating a patient, comprising:
placing an intubating laryngeal mask airway assembly, including a laryngeal mask airway and an endotracheal tube, into a lower pharynx of the patient;
separating the laryngeal mask airway into multiple components; and
independently removing each of the multiple components of the laryngeal mask airway from the patient while the endotracheal tube remains in place.

17. The method of claim 16, further including inflating a balloon of the endotracheal tube prior to independently removing each of the multiple components of the laryngeal mask airway from the patient.

18. The method of claim 17, further including ventilating the patient during a remainder of the intubating after inflation of the balloon.

19. The method of claim 16, further including ventilating the patient via the endotracheal tube simultaneous with independently removing each of the multiple components of the laryngeal mask airway from the patient.

20. The method of claim 16, wherein separating the laryngeal mask airway into multiple components includes:
removing a collar from an external end of a tubular sheath;
pulling opposing sides of the tubular sheath away from each other at the external end; and
sliding the opposing sides of the tubular sheath in opposing directions at an internal end.
